(19) United States
(12) Patent Application Publication
(10) Pub. No.: US 2016/0058753 A1
(43) Pub. Date: Mar. 3, 2016

(54) Nalmefene for Treatment of Patients with Mood Disorder

(71) Applicant: H. LUNDBECK A/S, Valby (DK)

(72) Inventors: Didier Meulien, Boulogne Billancourt (FR); David Gruhn, Valby (DK); Lars Torup, Værløse (DK); Björn Steiniger-Brach, Brønshøj (DK)

(73) Assignee: H. Lundbeck A/S, Valby (DK)

(21) Appl. No.: 14/784,723

(22) PCT Filed: Apr. 16, 2014
(86) PCT No.: PCT/EP2014/057678

§ 371 Date: Oct. 15, 2015

(30) Foreign Application Priority Data
Apr. 17, 2013 (DK) PA201300227

(51) Int. Cl. A61K 31/485; A61K 45/06

(52) U.S. Cl. CPC A61K 31/485; A61K 45/06

(57) ABSTRACT

The present invention relates to nalmefene for use in the treatment of mood disorders. The present invention further relates to nalmefene for use in the treatment of patients with alcohol dependence who have a co-morbid mood disorder. The invention further relates to nalmefene for use in the reduction of alcohol consumption in said patients. The invention further relates to nalmefene for use in the treatment of a mood disorder in said patients.

### FIELD OF THE INVENTION

The present invention relates to nalmefene for use in the treatment of mood disorders. The present invention further relates to nalmefene for use in the treatment of patients with alcohol dependence who have a co-morbid mood disorder. The invention further relates to nalmefene for use in the reduction of alcohol consumption in said patients. The invention further relates to nalmefene for use in the treatment of a mood disorder in said patients.

### BACKGROUND OF THE INVENTION

Nalmefene[17-(cyclopropylmethyl)-4,5-alpha-epoxy-6-methylenemorphinan-3,14-diol] has the following general formula:

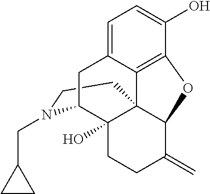

and can be prepared using methods that are well known in the art e.g. starting by manufacturing of naltrexone from noroxymorphone as described in WO 2012/059103 and subsequently manufacturing nalmefene from naltrexone e.g. by the Wittig reaction as described in WO 2010/136039.

Nalmefene is an opioid system modulator with a distinct μ, δ, and κ receptor profile. In vitro studies have demonstrated that nalmefene is a selective opioid receptor ligand with antagonist activity at the μ and δ receptors and partial agonist activity at the κ receptor. Acute alcohol intake was shown to result in mesolimbic dopamine release (facilitated by the release of β-endorphins), which can provide positive reinforcement. Nalmefene is thought to counteract the reinforcement effects and to reduce alcohol consumption, possibly by modulating these cortico-mesolimbic functions.

The efficacy and tolerability of nalmefene in the treatment of alcohol dependence have been evaluated in three phase III studies (two confirmatory 6-month efficacy studies and one 1-year safety study) conducted by Lundbeck (Mann et al. Extending the Treatment Options in Alcohol Dependence: A Randomized Controlled Study of As-Needed Nalmefene. Biol. Psychiatry (2013); 73: 706-713; Gual et al. A randomised, double-blind, placebo-controlled, efficacy study of nalmefene, as-needed use, in patients with alcohol dependence. European Neuropsychopharmacology (2013); 23(11):1432-1442; van den Brink et al., Long-term efficacy, tolerability and safety of nalmefene as-needed in patients with alcohol dependence: A 1-year, randomised controlled study. J. Psychopharmacol., published online before print Mar. 26, 2014, doi: 10.1177/0269881114527362) and 5 studies in alcohol use disorders conducted by the company Biotie (Karhuvaara et al. Alcohol. Clin Exp Res. (2007); 31: 1179-1187).

A marketing authorisation has recently been granted (February 2013) for oral nalmefene in the European Union (EU) under the tradename Selincro® for the reduction of alcohol consumption in adult patients with alcohol dependence.

Co-occurrence of alcohol dependence and depressive disorders are common and primarily based on findings from epidemiological studies which illustrate the complexity of the comorbidity between alcohol dependence on one side and mood disorders on the other side (Grant and Hartford, Drug and Alcohol Dependence, (1995), Vol. 39: 197-206.; Swendsen et al., Comprehensive Psychiatry, (1998), Vol. 38(4): 176-184; Swendsen and Merikangas, Clin. Psychol. Rev., (2000), Vol. 20(2):173-189; Kessler et al., Arch. Gen. Psychiatry, (1997), Vol. 54: 313-321).

These studies with often very large samples have also shown that there is a high level of lifetime comorbidity between depressive and anxiety disorders. Patients with depression have an increased risk of suffering from alcohol dependence compared to patients without depression. Likewise, patients with alcohol dependence have an increased risk of comorbid mood disorders compared to patients without alcohol dependence.

To illustrate this, Table 1 (Swendsen and Merikangas, Clin Psychol Rev., (2000), Vol. 20(2):173-189), presents the lifetime risks for different depressive disorders in patients with alcohol dependence compared to patients without alcohol dependence. Overall there was a 4.6 times higher risk of any mood disorder in patients with alcohol dependence and the risks for a particular depressive condition can vary: from a 2.8 times higher risk for dysthymia to a 8.1 times higher risk for bipolar disorder.

TABLE 1
Yale Family Study: Comorbidity of Alcoholism and Affective Disorders
in Relatives (Odds Ratios^{a} with 95% Confidence Limits)

| | Alcohol Dependence | |
|---|---|---|
| | versus None | |
| Disorder | Odds Ratio^{a} | 95% CI |
| Any mood disorder | 4.6 | [3.1; 6.9] |
| Bipolar disorder | 8.1 | [2.7; 24.0] |
| Major depression | 3.2 | [2.0; 5.0] |
| Dysthymia | 2.8 | [1.7; 4.8] |

^{a}adjusts for sex and comorbidity in probands and interview status, comorbidity, sex and age of relative
* p < 0.05;
** p < 0.001

When looking at the lifetime prevalence of comorbid alcohol dependence in patients with different types of mood disorders a complex picture also emerges. Patients with a major depressive episode have an almost 3 times higher lifetime risk of also suffering from alcohol dependence than patients without (>32% versus 11%) (Grant and Hartford, Drug and Alcohol Dependence, (1995), Vol. 39: 197-206).

Table 2 presents the lifetime co-occurrence of alcohol dependence in different mood disorders (lifetime diagnosis) based on data from the National Comorbidity Study (Kessler et al., Arch. Gen. Psychiatry, (1997), Vol. 54: 313-321). In patients with alcohol dependence there was a high lifetime prevalence of any depressive disorder; 28.1% in men and up to 53.5% in women. However, there is variation in lifetime prevalence for different types of depression. Furthermore, there is a possibility of suffering from more than one comorbid condition.

TABLE 2
Lifetime Co-occurrence of Alcohol Dependence with Other Lifetime
National Comorbidity Survey/DSM-III-R Disorders, by Sex.

| Lifetime | | | | | | |
|---|---|---|---|---|---|---|
| Disorders | Men (N = 806) | | | Women (N = 336) | | |
| Affective | %^{a} | OR^{b} | 95% CI | %^{a} | OR^{b} | 95% CI |
| Depres- | 24.3 | 2.95 | [2.21; 3.93] | 48.5 | 4.05 | [2.99; 5.48] |
| sion | | | | | | |
| Dysthymia | 11.2 | 3.81 | [2.37; 6.10] | 20.9 | 3.63 | [2.55; 5.18] |
| Mania | 6.2 | 12.03 | [4.79; 30.22] | 6.8 | 5.30 | [2.63; 10.69] |
| Any | 28.1 | 3.16 | [2.40; 4.16] | 53.5 | 4.36 | [3.30; 5.76] |

^{a}prevalences of the disorders represented in the rows among respondents with a lifetime diagnosis of alcohol dependence
^{b}all ORs (odds ratios) are significant at the 0.05 level, 2-tailed test

Comorbidity between alcohol dependence and mood disorder is also underlined by the various temporal patterns for the co-occurrence of these conditions, illustrated by the order of their onset such as primary, secondary or simultaneous onset. Table 3 (Swendsen et al., Comprehensive Psychiatry, (1998), Vol. 38(4): 176-184) shows that onset of depression disorder in relation to alcohol dependence can vary. For depression an onset before alcohol dependence is as frequent as a later onset.

TABLE 3
Retrospective Estimates for Order of Onset of Alcohol Abuse/Dependence
With Depressive Disorders.

| | Depressive Disorders* | | |
|---|---|---|---|
| Age of | | | Puerto |
| Onset | ECA | NCS | Rico |
| A < B | 45.0% | 54.9% | 40.0% |
| A = B | 10.0% | 10.7% | 20.0% |
| A > B | 45.0% | 34.4% | 40.0% |
| Totalno. | 174 | 441 | 15 |

Abbreviations:
A: alcoholism,
B: index disorder
*Any major depression or dysthymia.

Mood disorders and alcohol dependence carry a significant risk for the development of the other, and also the severity in one disorder is associated with severity in the other. The presence of depression has been reported to have an impact on the severity of alcohol dependence. On the other hand, the presence of alcohol dependence is associated with greater increases in the severity of depression, indicated by a higher number of symptoms (Swendsen and Merikangas, Clin. Psychol. Rev., (2000); Vol. 20(2):173-189), see Table 4.

TABLE 4
Comorbidity Between Alcoholism and Depressive Symptoms in the ECA
and NCS Studies and Symptom Severity Analysis

| | Increase in | Increase in |
|---|---|---|
| | Alcohol | Depressive |
| Investigation | Symptoms | Symptoms |
| ECA | 0.36* | 0.96* |
| NCS | 0.35* | 0.81* |

ECA = Epidemiological Catchment Area;
NCS = National Comorbidity Study
*p < 0.05

As described above, the co-occurrence is quite common. However the number of treatments for co-occurring mental and alcohol use disorders still remain low. It is explained historically by the fact that patients with alcohol use disorders were not prescribed medications because of the risks of potential drug interactions with alcohol or the risks for potential overdose of antidepressants and/or other mood disorder agents. It is also explained by the fact that the initial practice model that still remains has been in favour of sequential intervention, first treating the primary disorder initially then followed by treating the other disorder. However, this can result in delaying the treatment of one or the other comorbidity or not adequately assessing one of the comorbid disorder and lead to unwanted consequences. More recently it is now acknowledged that both disorders should be treated simultaneously and in an integrated and coordinated way (Pettinati et al., Am. J. Psychiatry (2013), Vol. 170: 23-30)

However, as stated in recent treatment guidelines (Lingford-Hughes et al., J. Psychopharmacol. (2012), Vol. 26(7): 899-952) “the antidepressants may improve mood but not necessarily substance use in those who are depressed with harmful or dependent substance use.” Generally, mood will only improve in those with a significant depressive disorder, and use of antidepressants should be restricted to this population and then with caution and monitored “In comorbid bipolar disorders there is mixed evidence of the efficacy for the existing pharmacological agents” (Lingford-Hughes et al., J. Psychopharmacol. (2012), Vol. 26(7): 899-952). Mood stabilizers such as lithium or valproate appear to be effective while antipsychotics such as quetiapine or aripiprazole are ineffective on measures of alcohol use and dependence (Stedman et al. Alcohol Clin Exp Res, (2010); 34(10): 1822-1831; Litten et al. Alcohol. Clin. Exp. Res. (2012), 36(3): 406-416; Anton et al. J. Clin. Psychopharmacol. (2008), 28: 5-12.)

Therefore there is a need for new treatments for use in patients with alcohol dependence who have a co-morbid mood disorder. In particular, there is a need for new treatments which could give rise to advantages such as e.g. improved efficacy and/or a different side effect profile compared to existing treatments.

### SUMMARY OF THE INVENTION

The present invention relates to nalmefene for use in the treatment of a mood disorder.

In one embodiment, the invention relates to nalmefene for use in the treatment of a patient with alcohol dependence who has a co-morbid mood disorder.

In one embodiment, the invention relates to nalmefene for use in the reduction of alcohol consumption in a patient with alcohol dependence who has a co-morbid mood disorder.

In one embodiment, the invention relates to a pharmaceutical composition comprising nalmefene and a second compound, which is a mood stabilizer; an antipsychotic agent suitable for treatment of bipolar disorders; or an antidepressant agent, and optionally acceptable carriers or diluents.

In one embodiment, the invention relates to a kit comprising nalmefene together with a second compound, which is a mood stabilizer; an antipsychotic agent suitable for treatment of bipolar disorders or an antidepressant agent.

In one embodiment, the invention relates to a method for the treatment of a mood disorder, which method comprises administering a pharmaceutically acceptable amount of nalmefene to a patient in need thereof.

In one embodiment, the invention relates to a method for reduction of alcohol consumption in a patient with alcohol dependence who has a co-morbid mood disorder, which method comprises administering a pharmaceutically acceptable amount of nalmefene to said patient.

In one embodiment, the invention relates to a method for reduction of alcohol consumption and for the treatment of a mood disorder, which method comprises administering a pharmaceutically acceptable amount of nalmefene to a patient in need thereof.

### BRIEF DESCRIPTION OF DRAWINGS

For all figures, -□- =placebo (PBO), -▪- =nalmefene (NMF), “B” denotes baseline. Number of patients “N” for placebo (PBO) and nalmefene (NMF), respectively throughout the study is indicated at the X-axis. Patients with and without a mood disorder at baseline were classified according to their ongoing medical history coded by the Medical Dictionary for Regulatory Activities (MedDRA).

FIGS. 1-2 show the change from baseline in monthly Heavy Drinking days (HDDs) and Total Alcohol Consumption (TAC) (g/day) in patients with a mood disorder at baseline vs. patients without a mood disorder at baseline.

FIGS. 1a-1b show the change from baseline in monthly HDDs. X-axis: time (months); Y-axis: change from baseline in mean HDD.

FIG. 1a: Patients without a mood disorder at baseline, change in monthly HDD.

FIG. 1b: Patients with a mood disorder at baseline, change in monthly HDD.

FIGS. 2a-2b show the change from baseline in monthly TAC (g/day). X-axis: time (months); Y-axis: change from baseline in mean TAC.

FIG. 2a: Patients without a mood disorder at baseline, change in monthly TAC.

FIG. 2b: Patients with a mood disorder at baseline, change in monthly TAC.

FIGS. 3-9 indicate change from baseline in POMS scores in patients with a mood disorder at baseline vs. patients without a mood disorder at baseline. X-axis: time (weeks); Y-axis: change from baseline in mean POMS.

FIG. 3a. Patients without a mood disorder at baseline, change in POMS total mood disturbance (TMD).

FIG. 3b: Patients with a mood disorder at baseline, change in POMS total mood disturbance (TMD).

FIG. 4a. Patients without a mood disorder at baseline, change in POMS Tension-Anxiety.

FIG. 4b: Patients with a mood disorder at baseline, change in POMS Tension-Anxiety.

FIG. 5a. Patients without a mood disorder at baseline, change in POMS Depression-Rejection.

FIG. 5b: Patients with a mood disorder at baseline, change in POMS Depression-Rejection.

FIG. 6a. Patients without a mood disorder at baseline, change in POMS Anger-Hostility.

FIG. 6b: Patients with a mood disorder at baseline, change in POMS Anger-Hostility.

FIG. 7a. Patients without a mood disorder at baseline, change in POMS Vigour.

FIG. 7b: Patients with a mood disorder at baseline, change in POMS Vigour.

FIG. 8a. Patients without a mood disorder at baseline, change in POMS Fatigue.

FIG. 8b: Patients with a mood disorder at baseline, change in POMS Fatigue.

FIG. 9a. Patients without a mood disorder at baseline, change in POMS Confusion.

FIG. 9b: Patients with a mood disorder at baseline, change in POMS Confusion.

### DEFINITIONS

Throughout the description, the term “nalmefene” is intended to include any form of the compound, such as the free base and pharmaceutically acceptable salts. The free base and pharmaceutically acceptable salts include anhydrous forms and solvated forms such as hydrates. The anhydrous forms and the solvates include amorphous and crystalline forms. In a particular embodiment, nalmefene is in the form of a hydrochloride salt. In a more particular embodiment, nalmefene is in the form of the hydrochloride dihydrate. Throughout the application, when a dose is specified for nalmefene, said dose is calculated as the free base, i.e. when the nalmefene dose is 18 mg this corresponds to 18 mg of nalmefene free base.

In the present context, the term “total alcohol consumption” abbreviated TAC indicates average total alcohol consumption measured in g/day

In the present context, the term “heavy drinking day” abbreviated HDD indicates a day with a total alcohol consumption ≧60 g of pure alcohol for men and ≧40 g for women.

In the present context, “as-needed dosing” indicates that on each day a patient perceives a risk of drinking alcohol, one dose of nalmefene should be taken, preferably 1-2 hours prior to the anticipated time of drinking. If the patient has started drinking alcohol without taking nalmefene, the patient should take one tablet as soon as possible after that.

As used herein, the term “drinking risk level” abbreviated DRL is defined according to the criterias defined by the World Health Organization in “International Guide for Monitoring Alcohol Consumption and Related Harm” (2000), WHO, as outlined in Table 1 below.

TABLE 5
WHO Drinking Risk Levels (DRLs) of Alcohol Consumption

Total Alcohol
Consumption (g/day)

| DRL | Men | Women |
|---|---|---|
| Very high risk | >100 | >60 |
| High risk | >60 to 100 | >40 to 60 |
| Medium risk | >40 to 60 | >20 to 40 |
| Low risk | 1 to 40 | 1 to 20 |

Drinking Risk Levels according to Table 5 can be assessed e.g. by calculating mean daily alcohol consumption in g/day over a period such as 1 week or longer, such as 2 weeks or longer, such as 3 weeks or longer, such as 4 weeks or longer, such as 1 month or longer such as 2 months or longer, such as 3 months or longer, such as 4 months or longer, such as 5 months or longer, such as 6 months or longer, such as about 1 year. Assessment of DRL can be performed by specialists and/or physicians such as general practitioners and/or other health care providers based on patients estimates of their alcohol consumption.

Throughout the application, the term “high risk” or “at least high risk” is intended to include the two groups defined as “high risk” and “very high risk” according to WHOs drinking risk levels listed in Table 5, i.e. patients having drinking risk level corresponding to a total alcohol consumption of >60 g/day of pure alcohol for men and >40 g/day for women. The present invention does not distinguish between patients with high and very high drinking risk levels, and when the terms “high drinking risk level” or “high DRL” are used in a claim or in an embodiment of the invention it is intended to include both the group defined as “high risk” and the group defined as “very high risk” according to WHOs drinking risk levels listed in Table 5.

As used herein, the terms “motivational support” and “counselling focused on enhanced treatment adherence and reduced alcohol consumption” indicate psychological motivation-enhancing interventions and can be used interchangeably with the terms “psychosocial support” or “psychosocial intervention focused on treatment adherence and reducing alcohol consumption”. Said motivational support can be administered by a specialist and/or a physician such as a general practitioner and/or other health care providers. One example of such interventions is the BRENDA model, which is a time-limited, patient-centered clinical motivational intervention that complements the use of medication with focus on changing behavior and increasing medication adherence. The BRENDA model has been described by Starosta et al., J. Psychiatr. Pract. (2006), Vol. 12(2): 80-89, the entire contents of which are incorporated herein by reference. The term “initial motivational support” indicates such motivation enhancing interventions provided to the patient prior to treatment with nalmefene. The term “ongoing motivational support” indicates such motivation-enhancing interventions provided to the patient concurrent to treatment with nalmefene e.g. on a recurrent basis.

In the present context, “Pharmaceutical composition” refers to a dose form such as an oral dose form, such as a solid oral dose form, typically tablets or capsules. “Pharmaceutical compositions of the present invention” refers to all pharmaceutical compositions covered by the claims and description.

In the present context, a “unit dosage form” refers to a formulation unit of a pharmaceutical composition e.g. one tablet or capsule.

In the present context, “therapeutically effective amount” of a compound means the amount/dose of a compound or pharmaceutical composition that is sufficient to produce an effective response (i.e., a biological or medical response of a tissue, system, animal or human sought by a researcher, veterinarian, medical doctor or other clinician) upon administration to a patient. The “therapeutically effective amount” will vary depending on, inter alia, the disease and its severity, and on the age, weight, physical condition and responsiveness of the patient to be treated. Furthermore, the “therapeutically effective amount” may vary if nalmefene is combined with one or more other compounds: In such a case the amount of a given compound might be lower, such as a sub-effective amount.

In the present context, “treatment” and “treating” refers to the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relieve the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. In one aspect of the present invention, “treatment” and “treating” refers to prophylactic (preventive) treatment. In another aspect, “treatment” and “treating” refers to curative treatment. The patient to be treated is preferably a mammal, in particular a human being.

The term “alcohol dependence” is a commonly known term for a skilled person and is e.g. described in the revised 4^{th} edition of the Diagnostic and Statistical Manual of Mental Disorders (DSM-IV-TR) (Diagnostic and Statistical Manual of Mental Disorders, 4^{th} edition text revision, American Psychiatric Publishing, 2000). As used herein, the term “alcohol dependence” is defined as the presence of three or more of the seven areas of life impairment related to alcohol in the same 12-month period. These impairments include 1) tolerance, 2) withdrawal, 3) the alcohol is often taken in larger amounts or over a longer period than was intended, 4) persistent desire or unsuccessful efforts to cut down or control alcohol intake, 5) a great deal of time is spent in activities necessary to obtain alcohol, intake alcohol, or recover from its effects, 6) important social, occupational, or recreational activities are given up or reduced because of alcohol consumption, 7) alcohol use is continued despite knowledge of having a persistent or recurrent physical or psychological problem that is likely to have been caused or exacerbated by alcohol consumption.

The term “mood disorder” is described in DSM-IV-TR and refers to a variety of conditions characterized by a disturbance in mood as the main feature. In the present context, mood disorders include major depressive disorder, dysthymic disorder, depressive disorder not otherwise specified, bipolar I disorder, bipolar II disorder, cyclothymic disorder and bipolar disorder not otherwise specified.

In the present context, “patients with co-morbid mood disorder” refers to patients who are alcohol dependent and at the same time have a mood disorder. In one embodiment, said mood disorder is caused by said alcohol dependence e.g. said mood disorder is an alcoholinduced mood disorder. In one embodiment, said alcohol dependence is caused by said mood disorder. In one embodiment said alcohol dependence and said mood disorder are not causally related to each other.

The term “alcohol induced mood disorder” is described in DSM-IV-TR and refers to a disorder characterized by prominent and persistent disturbance in mood that is judged to be a direct physiological consequence of alcohol abuse.

The term “selective serotonin reuptake inhibitor” (SSRI) means an inhibitor of the monoamine transporters which has stronger effect at the serotonin transporter than at the dopamine and the noradrenaline transporters.

The term “serotonin and noradrenaline reuptake inhibitor” (SNRI) means an inhibitor of the monoamine transporters which has an effect both at the serotonin transporter and at the noradrenaline transporter.

The term “POMS” is an abbreviation of “profile of mood states” and refers to a selfreport inventory scale developed to assess the effect of e.g. new medication on mood states and mood changes. The scale measures six domains: Tension-Anxiety, Depression-Rejection, Anger-Hostility, Vigour-Activity, Fatigue-Inertia, and Confusion-Bewilderment. A total mood disturbance (TMD) score can be calculated. In general, a lower POMS score indicates a better mood state than a higher score except for vigour-activity, for which a higher POMS score indicates a better mood state. The scale has been described e.g. by McNair et al., Profile of mood states. San Diego, Calif.: Educational and Industrial Testing Service and by Nyenhios and Yamamoto, J. Clin. Psychology, (1999), Vol. 55(1): 79-86.

“MedDRA” is an abbreviation of Medical Dictionary for Regulatory Activities which is a clinically validated international medical terminology dictionary (and thesaurus) used by regulatory authorities in the pharmaceutical industry during the regulatory process, from premarketing to post-marketing activities, and for data entry, retrieval, evaluation, and presentation. In addition, it is the adverse event classification dictionary endorsed by the International Conference on Harmonisation of Technical Requirements for Registration of Pharmaceuticals for Human Use (ICH).

### DETAILED DESCRIPTION OF THE INVENTION

The efficacy of nalmefene in the reduction of alcohol consumption in patients with alcohol dependence (DSM-IV) has been evaluated in study 12013A. The efficacy of nalmefene was measured using two co-primary endpoints: the change from baseline in the monthly number of heavy drinking days (HDDs) and the change from baseline in the average daily total alcohol consumption (TAC). In the total patient group, nalmefene was superior to placebo in reducing the number of HDDs and in reducing TAC.

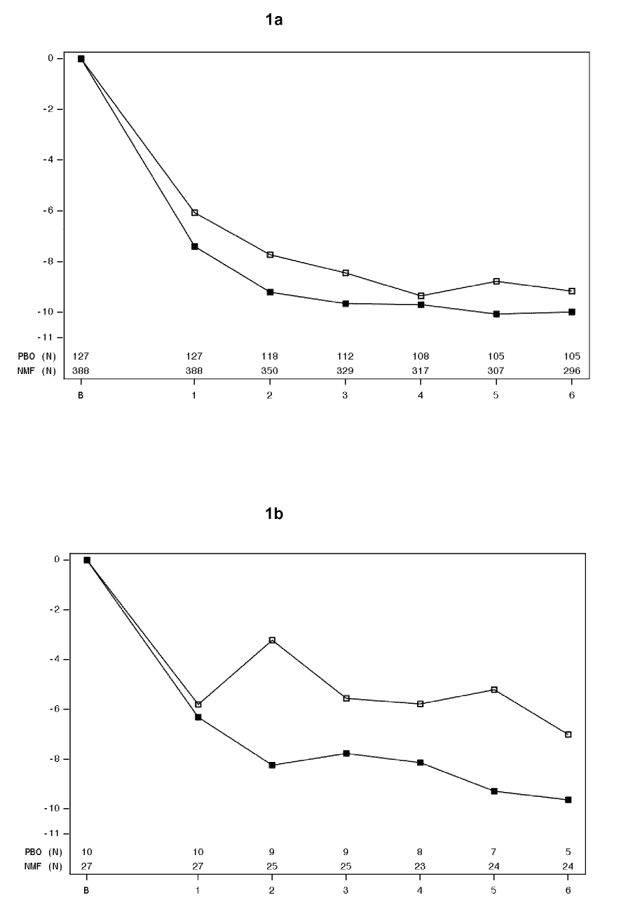

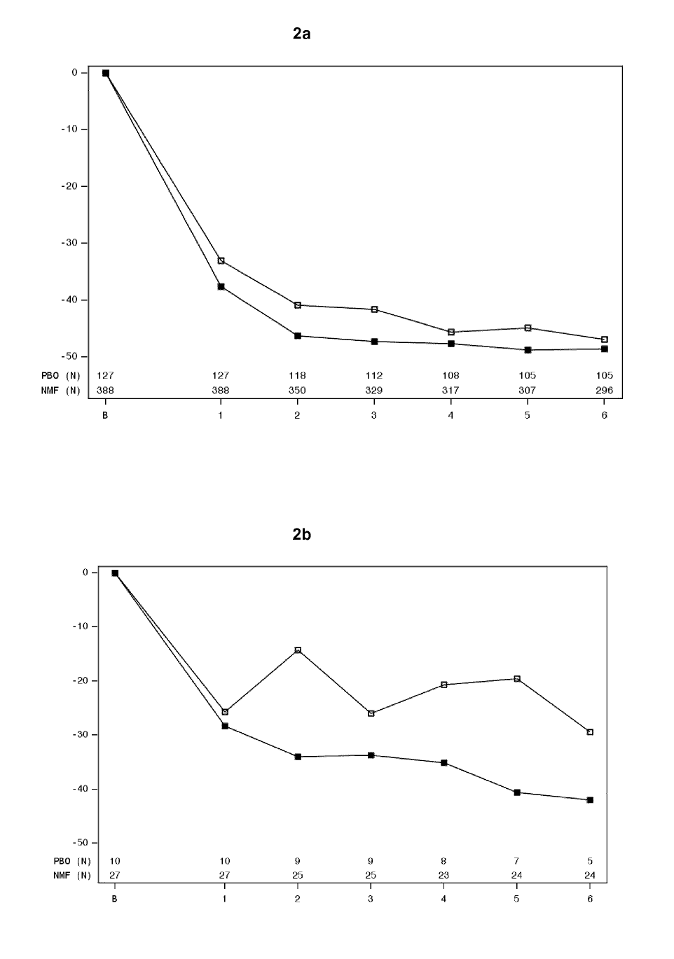

The inventors have found that nalmefene significally reduced the alcohol consumption in patients with a mood disorder at baseline. The effect of nalmefene on both HDDs and TAC in this patient group was more pronounced compared to placebo than in patients without a mood disorder at baseline i.e. nalmefene has a better effect on the reduction of alcohol consumption in patients with a mood disorder at baseline than in patients without a mood disorder at baseline (FIGS. 1-2). Therefore, in one embodiment, the present invention relates to nalmefene for use in the treatment of patients with alcohol dependence who have a comorbid mood disorder. In one embodiment, the present invention relates to nalmefene for use in the reduction of alcohol consumption in patients with alcohol dependence who have a comorbid mood disorder.

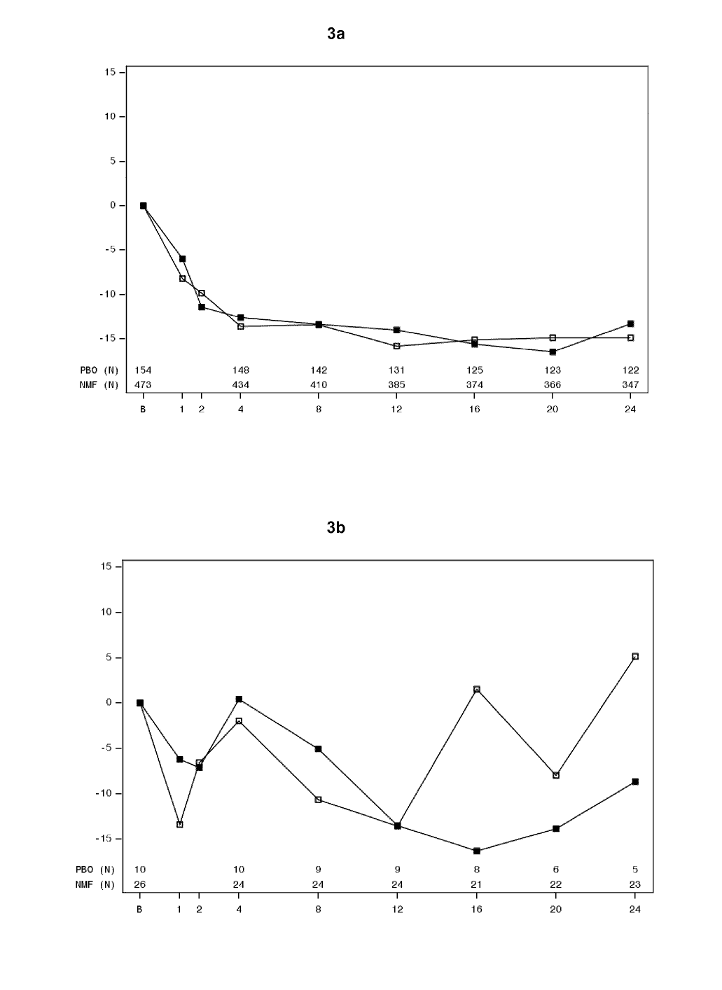

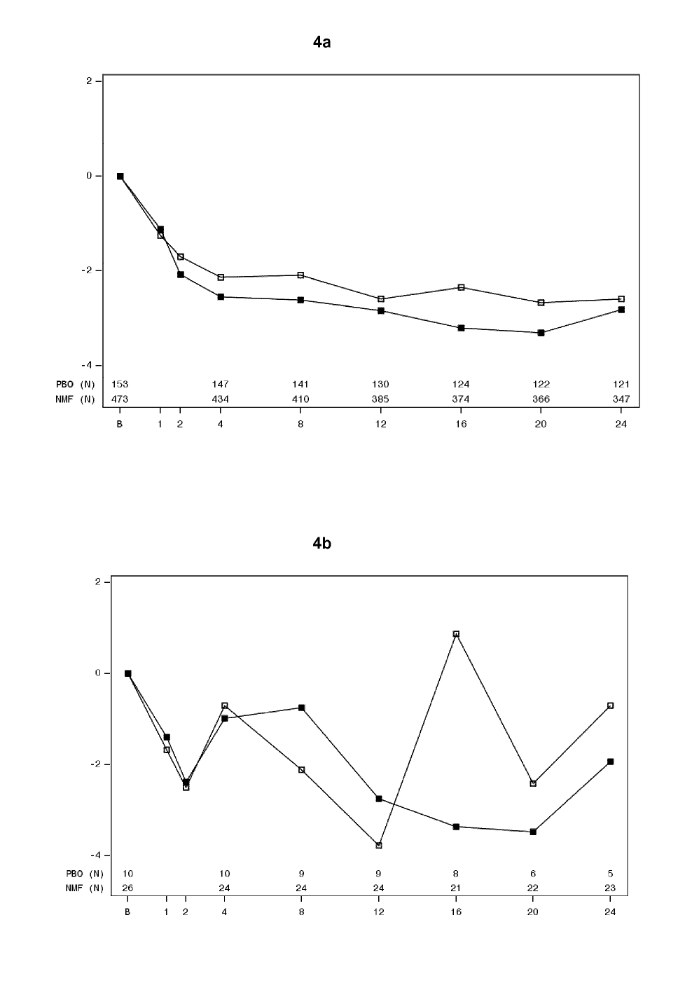

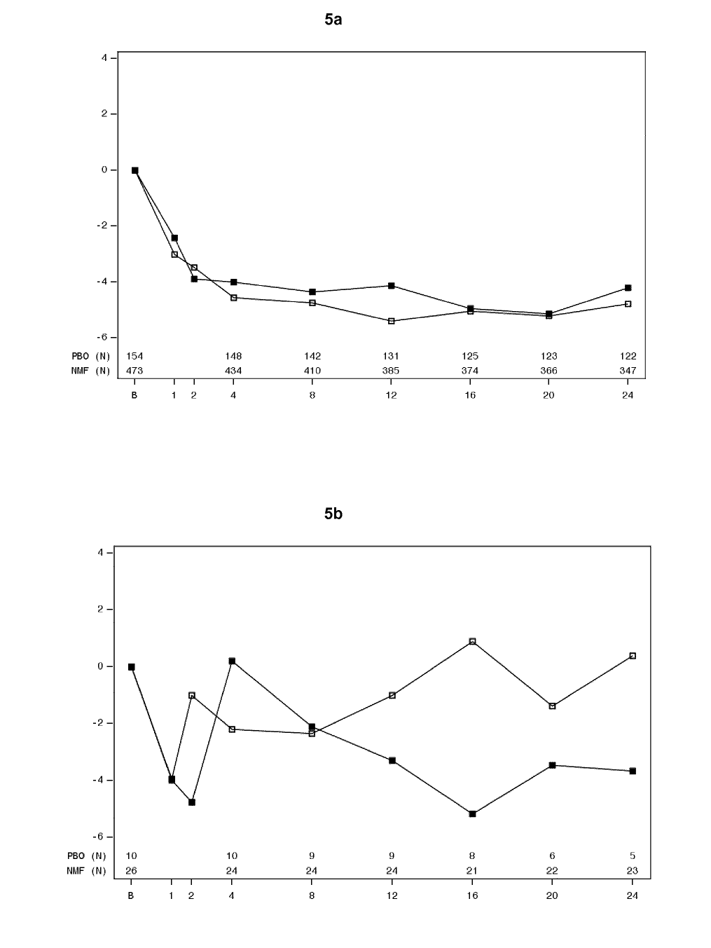

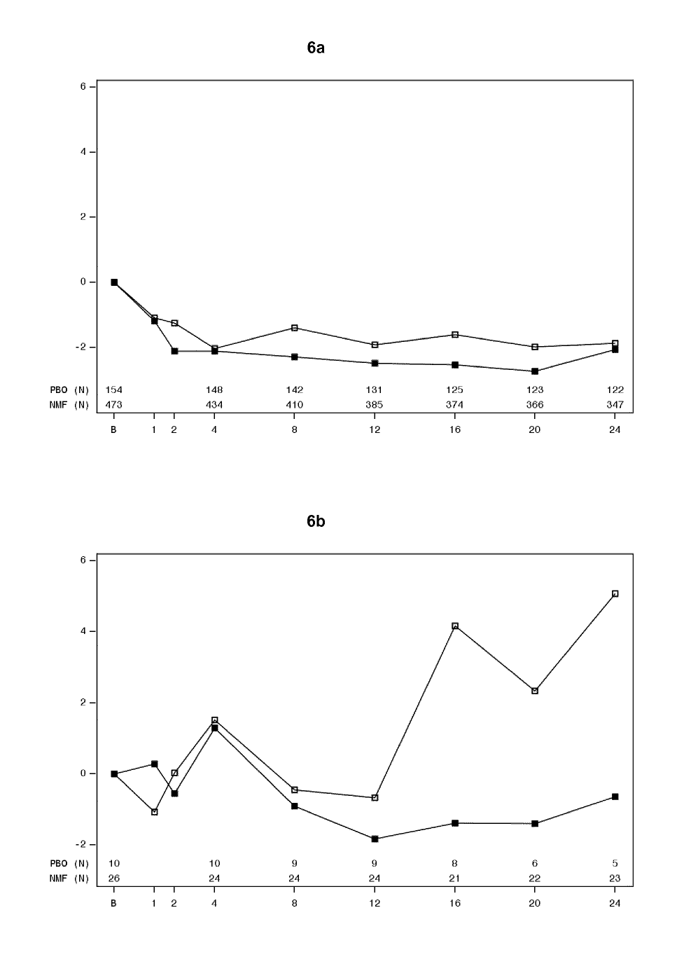

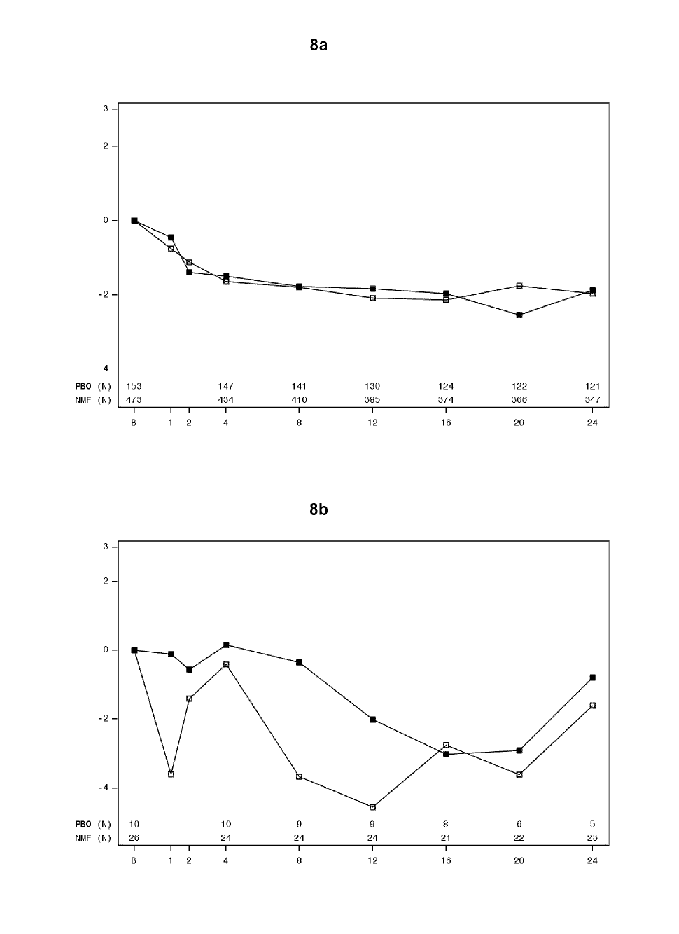

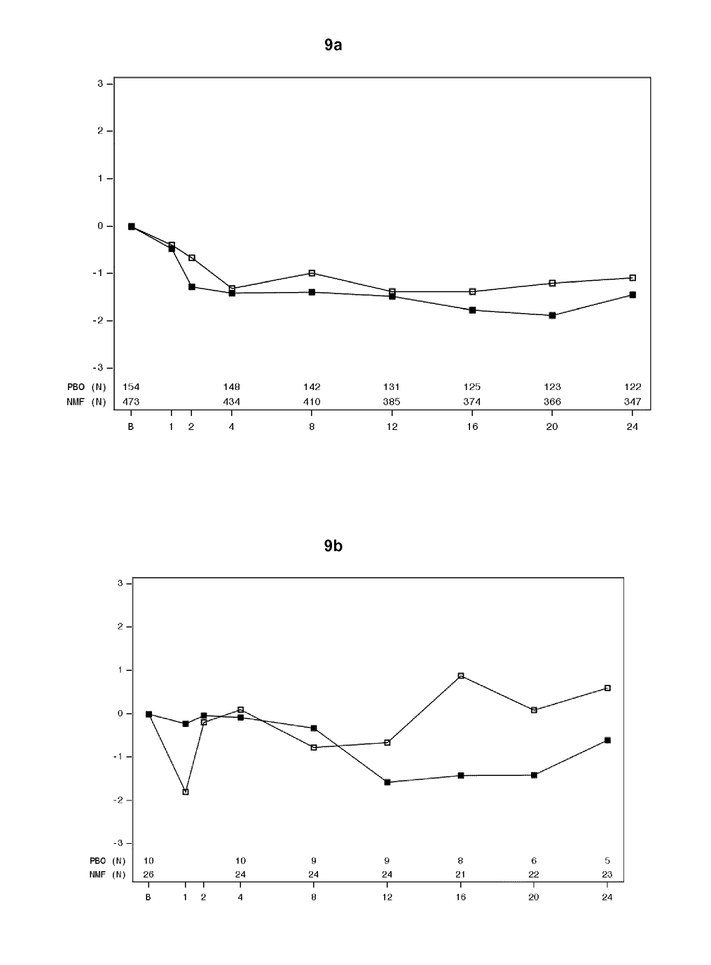

Assessment of POMs scores in Study 12013A was used to evaluate the effect of nalmefene on mood states and mood changes throughout the study. The inventors of the present invention surprisingly found that nalmefene has an effect on the POMS scores in patients with a mood disorder. Tables 7 and 9 indicate that patients with a mood disorder at baseline had higher POMS scores at baseline when compared to those patients without mood disorders. The change in POMS scores from baseline are illustrated in FIGS. 3-9. FIGS. 3a-9a indicates that in patients without a mood disorder at baseline, the pattern in POMS score was stabile throughout the study with no pronounced difference between nalmefene and placebo. FIGS. 3b-9b indicates that the patients with a mood disorder at baseline who received nalmefene had a better POMS score at the end of the study than the patients with a mood disorder at baseline who received placebo. In particular FIGS. 3b, 4b, 5b, 6b and 9b representing total mood disturbance, tension-anxiety, depression-rejection, anger-hostility and confusion, respectively, indicates better POMS scores in weeks 16-24 in patients who received nalmefene compared to patients who received placebo. Overall, the POMS data indicates that the general mood state improves in patients with a mood disorder when said patients are treated with nalmefene.

Accordingly, in one embodiment, the present invention therefore relates to nalmefene for treatment of a mood disorder. In one embodiment, the invention relates to nalmefene for treatment of a mood disorder in patients with alcohol dependence who have a co-morbid mood disorder. In a further embodiment, the invention relates to nalmefene for use in the reduction of alcohol consumption and for treatment of a mood disorder in patients with alcohol dependence who have a co-morbid mood disorder.

In one embodiment, nalmefene is used as the sole active ingredient for the treatment of a mood disorder. In one embodiment, nalmefene is used as the sole active ingredient in the treatment of patients with alcohol dependence who have a co-morbid mood disorder.

In one embodiment, nalmefene is used in combination with a second compound which is selected from a mood stabilizer; an antipsychotic agent suitable for treatment of bipolar disorders; or an antidepressant agent such as a serotonin reuptake inhibitor, or any other compound which causes an elevation in the level of extracellular serotonin for the treatment of a mood disorder. In another embodiment, nalmefene is used in combination with a second compound which is selected from a mood stabilizer; an antipsychotic agent suitable for treatment of bipolar disorders; or an antidepressant agent such as a serotonin reuptake inhibitor, or any other compound which causes an elevation in the level of extracellular serotonin in the treatment of patients with alcohol dependence who have a co-morbid mood disorder.

The present invention also relates to a pharmaceutical composition comprising nalmefene and a second compound, which is selected from a mood stabilizer; an antipsychotic agent suitable for treatment of bipolar disorders; or an antidepressant agent such as a serotonin reuptake inhibitor, or any other compound which causes an elevation in the level of extracellular serotonin, and optionally acceptable carriers or diluents.

Further assessment of the effect of nalmefene on the treatment of mood disorders can be performed by testing nalmefene in non-clinical models e.g. acute models such as the forced swim test model, and/or the marble burying model as outlined in Examples 4-6 or chronic models e.g. such as the chronic mild stress model described in Example 7. In such models nalmefene can be tested as the sole active substance as well as in combination with other compounds.

According to the present invention, nalmefene or a pharmaceutically acceptable salt thereof may be administered in any suitable way, e.g. orally, transmucosally or parenterally, and it may be presented in any suitable form for such administration, e.g. in the form of tablets, capsules, powders, syrups or solutions or dispersions for injection. In another embodiment, and in accordance with the purpose of the present invention, nalmefene is administered in the form of a solid pharmaceutical entity, suitably as a tablet or a capsule or in the form of a suspension, solution or dispersion for injection. Additionally, nalmefene may be administered with a pharmaceutically acceptable carrier, such as an adjuvant and/or diluent.

Methods for the preparation of solid or liquid pharmaceutical preparations are well known in the art. See e.g. Remington: The Science and Practice of Pharmacy, 21^{st} ed., Lippincott Williams & Wilkins (2005). Tablets may thus be prepared by mixing the active ingredients with an ordinary carrier, such as an adjuvant and/or diluent, and subsequently compressing the mixture in a tableting machine. Non-limiting examples of adjuvants and/or diluents include: corn starch, lactose, talcum, magnesium stearate, gelatin, lactose, gums, and the like. Any other adjuvant or additive such as colorings, aroma, and preservatives may also be used provided that they are compatible with the active ingredients. The pharmaceutical compositions of the invention thus typically comprise an effective amount of nalmefene and one or more pharmaceutically acceptable carrier. A suitable oral formulation of nalmefene is described in WO 2012/059103.

Without limiting the invention in any way, it is intended that any one of the aspects or embodiments of this patent application is suitable for the medicaments or pharmaceutical compositions described herein.

Nalmefene may be administered as an oral dose form, such as a solid oral dose form, typically tablets or capsules, or as a liquid oral dose form. Nalmefene may be administered in an immediate release dosage form or a controlled or sustained release dosage form. Nalmefene may be conveniently administered orally in unit dosage forms, such as tablets or capsules, containing the active ingredient in an amount from about 1 to about 100 mg, such as from 5 to 50 mg. Typically, the pharmaceutical composition comprises from 10 mg to 20 mg, such as about 10 mg, about 11 mg, about 12 mg, about 13 mg, about 14 mg, about 15 mg, about 16 mg, about 17 mg, about 18 mg, about 19 mg or about 20 mg of nalmefene. In a preferred embodiment, the pharmaceutical composition comprises about 18 mg of nalmefene. In one embodiment, the unit dosage form comprises nalmefene in a therapeutically effective amount.

In one embodiment, nalmefene is taken as-needed, that is, on each day a patient perceives a risk of drinking alcohol, one dose of nalmefene should be taken, preferably 1-2 hours prior to anticipated time of drinking. In one embodiment, if the patient has started drinking alcohol without taking nalmefene, the patient should take one dose of nalmefene as soon as possible after that.

Nalmefene according to the present invention is intended to be used for dosing in humans who are adults or adolescents.

In one embodiment, nalmefene is in the form of the hydrochloride dihydrate.

According to the invention, nalmefene can be used in combination with a second compound which is a mood stabilizer; an antipsychotic agent suitable for treatment of bipolar disorders; or an antidepressant agent such as a serotonin reuptake inhibitor, or any other compound which causes an elevation in the level of extracellular serotonin. Said mood stabilizer may e.g. be selected from the following compounds; lithium, valproic acid, lamotrigine, carbamazepine, oxcarbazepine, topiramate, riluzole, gabapentin. Said antipsychotic agent may e.g. be selected from the following compounds; olanzapine, aripiprazole, quetiapine, risperidone, ziprasidone, asenapine. Said antidepressant agent may e.g. be selected from the following compounds; citalopram, escitalopram, fluoxetine, sertraline, paroxetine, fluvoxamine, venlafaxine, duloxetine, dapoxetine, nefazodone, imipramine, femoxetine, clomipramine, agomelatine, mirtazapine. The compounds mentioned above may be used in the form of the base or a pharmaceutically acceptable salt, such as an acid addition salt, thereof.

The above list of mood stabilizers; antipsychotic agents suitable for treatment of bipolar disorders; and antidepressant agents may not be construed as limiting.

Mood stabilizers, antipsychotic agents and antidepressant agents including serotonin reuptake inhibitors, including the SNRIs, SSRIs and other agents specifically mentioned hereinabove, differ both in molecular weight and in activity. As a consequence, the amount of said second compound used in combination therapy depends on the nature of said second compound. In one embodiment of the invention, said second compound is administered at lower doses than required when the compound is used alone. In another embodiment, said second compound is administered in normal therapeutic doses.

To prepare the pharmaceutical compositions of this invention, an appropriate amount of the active ingredient(s), in salt form or base form, is combined in an intimate admixture with a pharmaceutically acceptable carrier, which can take a wide variety of forms depending on the form of preparation desired for administration. These pharmaceutical compositions are desirably in unitary dosage form suitable for administration orally, rectally, percutaneously or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed.

It is especially advantageous to formulate the aforementioned pharmaceutical compositions in dosage unit form for ease of administration and uniformity of dosage. As used in the specification and claims, unit dosage form refers to physically discrete units suitable as unitary dosages, each unit containing a predetermined quantity of active ingredient(s) calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Nalmefene may be administered before, during or after the administration of said second compound provided that the time between the administration of nalmefene and the administration of said second compound is such that ingredients are allowed to act synergistically on the CNS. When simultaneous administration of nalmefene and said second compound is envisaged, a composition containing both said second compound and nalmefene may be particularly convenient. Alternatively, nalmefene and said second compound may be administered separately in the form of suitable compositions. The compositions may be prepared as described hereinabove.

The present invention also comprises products containing nalmefene and a second compound which is a mood stabilizer; an antipsychotic agent suitable for treatment of bipolar disorders; or an antidepressant agent such as a serotonin reuptake inhibitor, or any other compound which causes an elevation in the level of extracellular serotonin; as a combination preparation for simultaneous, separate or sequential use in drug therapy. Such products may comprise, for example, a kit comprising discrete unit dosage forms containing nalmefene and discrete unit dosage forms containing said second compound, all contained in the same container or pack, e.g. a blister pack.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the various aspects, embodiments, implementations and features of the invention mentioned herein may be claimed separately, or in any combination.

### EMBODIMENTS ACCORDING TO THE INVENTION

In the following, embodiments of the invention are disclosed. The first embodiment is denoted E1, the second embodiment is denoted E2 and so forth.
E1. Nalmefene for use in the treatment of a mood disorder.
E2. Nalmefene for use in the treatment of a patient with alcohol dependence who has a co-morbid mood disorder.
E3. Nalmefene for use in the reduction of alcohol consumption in a patient with alcohol dependence who has a co-morbid mood disorder.
E4. Nalmefene according to embodiment 1 or 2 for use in the treatment of a mood disorder in a patient with alcohol dependence who has a co-morbid mood disorder.
E5. Nalmefene for use in the reduction of alcohol consumption according to embodiment 3 and for use in the treatment of a mood disorder according to embodiment 4 in a patient with alcohol dependence who has a co-morbid mood disorder.
E6. Nalmefene according to any of embodiments 1-5, wherein said mood disorder or comorbid mood disorder is an alcohol induced mood disorder.
E7. Nalmefene according to any of embodiments 2-5, wherein said alcohol dependence is caused by said mood disorder.
E8. Nalmefene according to any of embodiments 2-5, wherein said alcohol dependence and said mood disorder are not causally related to each other.
E9. Nalmefene according to any of embodiments 1-8, wherein said mood disorder or comorbid mood disorder is selected from major depressive disorder, dysthymic disorder, depressive disorder not otherwise specified, bipolar I disorder, bipolar II disorder, cyclothymic disorder and bipolar disorder not otherwise specified.
E10. Nalmefene according to any of embodiments 1-9, wherein said nalmefene is the sole active ingredient used in the treatment of said mood disorder and/or in the reduction of said alcohol consumption.
E11. Nalmefene according to any of embodiments 1-10, wherein said patient is further treated with a second compound which is a mood stabilizer; an antipsychotic agent suitable for treatment of bipolar disorders; or an antidepressant agent.
E12. Nalmefene according to embodiment 11, wherein said second compound is a mood stabilizer.
E13. Nalmefene according to embodiment 12, wherein said mood stabilizer is selected from lithium, valproic acid, lamotrigine, carbamazepine, oxcarbazepine, topiramate, riluzole and gabapentin, or a pharmaceutically acceptable salt of any of these compounds.
E14. Nalmefene according to embodiment 11, wherein said second compound is an antipsychotic agent suitable for treatment of bipolar disorders.
E15. Nalmefene according to embodiment 14, wherein said antipsychotic agent suitable for treatment of bipolar disorders is selected from olanzapine, aripiprazole, quietapine, risperidone, ziprasidone or asenapine, or a pharmaceutically acceptable salt of any of these compounds.
E16. Nalmefene according to embodiment 11, wherein said second compound is an antidepressant agent.
E17. Nalmefene according to embodiment 16, wherein said antidepressant agent is a serotonin reuptake inhibitor, or any other compound which causes an elevation in the level of extracellular serotonin.
E18. Nalmefene according to embodiment 17, wherein said serotonin reuptake inhibitor is a selective serotonin reuptake inhibitor.
E19. Nalmefene according to embodiment 16, wherein said antidepressant agent is selected from citalopram, escitalopram, fluoxetine, sertraline, paroxetine, fluvoxamine, venlafaxine, duloxetine, dapoxetine, nefazodone, imipramine, femoxetine, clomipramine, agomelatine, or a pharmaceutically acceptable salt of any of these compounds.
E20. Nalmefene according to any of embodiments 11-19, wherein said nalmefene and said second compound are contained in the same unit dosage form.
E21. Nalmefene according to any of embodiments 11-19, wherein said nalmefene and said second compound are contained in separate unit dosage forms.
E22. Nalmefene according to any of embodiments 2-21, wherein said patient has at least a medium drinking risk level.
E23. Nalmefene according to embodiment 22, wherein said patient has a high drinking risk level.
E24. Nalmefene according to embodiment 23, wherein said patient has a drinking risk level corresponding to consumption >60 g/day of pure alcohol for men and >40 g/day for women.
E25. Nalmefene according to any of embodiments 1-24, wherein said nalmefene is to be used as-needed.
E26. Nalmefene according to any of embodiments 1-25, wherein said nalmefene is used in a dose of 10-20 mg such as 10 mg, 11 mg, 12 mg, 13 mg, 14 mg, 15 mg, 16 mg, 17 mg, 18 mg, 19 mg or 20 mg.
E27. Nalmefene according to embodiment 26, wherein said nalmefene is used in a dose of 18 mg.
E28. Nalmefene according to any of embodiments 1-27, wherein said nalmefene is used in the form of a pharmaceutically acceptable acid addition salt.
E29. Nalmefene according to embodiment 28, wherein said nalmefene is used in the form of a hydrochloride salt.
E30. Nalmefene according to embodiment 29, wherein said nalmefene is used in the form of the hydrochloride dihydrate.
E31. Nalmefene according to embodiment 30, wherein said nalmefene is used in a crystalline form.
E32. Nalmefene according to any of embodiments 1-31, wherein said nalmefene is contained in an oral dose form such as tablets or capsules.
E33. A pharmaceutical composition comprising nalmefene and a second compound which is a mood stabilizer; an antipsychotic agent suitable for treatment of bipolar disorders; or an antidepressant agent, and optionally acceptable carriers or diluents.
E34. A kit comprising nalmefene together with a second compound, which is a mood stabilizer; an antipsychotic agent suitable for treatment of bipolar disorders; or an antidepressant agent.
E35. The pharmaceutical composition according to embodiment 33 or the kit according to embodiment 34, wherein said second compound is a mood stabilizer.
E36. The pharmaceutical composition or the kit according to embodiment 35, wherein said mood stabilizer is selected from lithium, valproic acid, lamotrigine, carbamazepine, oxcarbazepine, topiramate, riluzole and gabapentin, or a pharmaceutically acceptable salt of any of these compounds.
E37. The pharmaceutical composition according to embodiment 33 or the kit according to embodiment 34, wherein said second compound is an antipsychotic agent suitable for treatment of bipolar disorders.
E38. The pharmaceutical composition or the kit according to embodiment 37, wherein said antipsychotic agent suitable for treatment of bipolar disorders is selected from olanzapine, aripiprazole, quietapine, risperidone, ziprasidone or asenapine, or a pharmaceutically acceptable salt of any of these compounds.
E39. The pharmaceutical composition according to embodiment 33 or the kit according to embodiment 34, wherein said second compound is an antidepressant agent.
E40. The pharmaceutical composition or the kit according to embodiment 39, wherein said antidepressant agent is a serotonin reuptake inhibitor, or any other compound which causes an elevation in the level of extracellular serotonin.
E41. The pharmaceutical composition or the kit according to embodiment 40, wherein said serotonin reuptake inhibitor is a selective serotonin reuptake inhibitor.
E42. The pharmaceutical composition or the kit according to embodiment 39, wherein said antidepressant agent is selected from citalopram, escitalopram, fluoxetine, sertraline, paroxetine, fluvoxamine, venlafaxine, duloxetine, dapoxetine, nefazodone, imipramine, femoxetine, clomipramine, agomelatine, or a pharmaceutically acceptable salt of any of these compounds.
E43. The kit according to any of embodiments 34-42, which is adapted for sequential administration of said nalmefene and said second compound.
E44. The pharmaceutical composition or the kit according to any of embodiments 33-42, which is adapted for simultaneous administration of said nalmefene and said second compound.
E45. The pharmaceutical composition or the kit according to embodiment 44, wherein said nalmefene and said second compound are contained in the same unit dosage form.
E46. The pharmaceutical composition or the kit according to any of embodiments 33-45, wherein said nalmefene is present in a dose of 10-20 mg such as 10 mg, 11 mg, 12 mg, 13 mg, 14 mg, 15 mg, 16 mg, 17 mg, 18 mg, 19 mg or 20 mg.
E47. The pharmaceutical composition or the kit according to embodiment 46, wherein said nalmefene is present in a dose of 18 mg.
E48. The pharmaceutical composition or the kit according to any of embodiments 33-47, wherein said nalmefene is present in the form of a pharmaceutically acceptable acid addition salt.
E49. The pharmaceutical composition or the kit according to embodiment 48, wherein said nalmefene is present in the form of a hydrochloride salt.
E50. The pharmaceutical composition or the kit according to embodiment 49, wherein said nalmefene is present in the form of the hydrochloride dihydrate.
E51. The pharmaceutical composition or the kit according to embodiment 50, wherein said nalmefene is present in a crystalline form.
E52. A method for the treatment of a mood disorder, which method comprises administering a pharmaceutically acceptable amount of nalmefene to a patient in need thereof.
E53. A method for reduction of alcohol consumption in a patient with alcohol dependence who has a co-morbid mood disorder, which method comprises administering a pharmaceutically acceptable amount of nalmefene to said patient.
E54. The method according to embodiment 52 wherein said patient is alcohol dependent and has a co-morbid mood disorder.
E55. A method for reduction of alcohol consumption and for the treatment of a mood disorder, which method comprises administering a pharmaceutically acceptable amount of nalmefene to a patient in need thereof.
E56. The method according to embodiment 55, wherein said patient is alcohol dependent and has a co-morbid mood disorder.
E57. The method according to any of embodiments 52-56, wherein said mood disorder or co-morbid mood disorder is an alcohol induced mood disorder.
E58. The method according to any of embodiments 52-56, wherein said alcohol dependence is caused by said mood disorder.
E59. The method according to any of embodiments 52-56, wherein said alcohol dependence and said mood disorder are not causally related to each other.
E60. The method according to any of embodiments 52-59, wherein said mood disorder or co-morbid mood disorder is selected from major depressive disorder, dysthymic disorder, depressive disorder not otherwise specified, bipolar I disorder, bipolar II disorder, cyclothymic disorder and bipolar disorder not otherwise specified.
E61. The method according to any of embodiments 52-60, wherein said nalmefene is the sole active ingredient used in the treatment of said mood disorder and/or in the reduction of said alcohol consumption.
E62. The method according to any of embodiments 62-60, which method further comprises administering a pharmaceutically acceptable amount of a second compound which is a mood stabilizer; an antipsychotic agent suitable for treatment of bipolar disorders; or an antidepressant agent.
E63. The method according to embodiment 62, wherein said second compound is a mood stabilizer.
E64. The method according to embodiment 63, wherein said mood stabilizer is selected from lithium, valproic acid, lamotrigine, carbamazepine, oxcarbazepine, topiramate, riluzole and gabapentin, or a pharmaceutically acceptable salt of any of these compounds.
E65. The method according to embodiment 62, wherein said second compound is an antipsychotic agent suitable for treatment of bipolar disorders.
E66. The method according to embodiment 65, wherein said antipsychotic agent suitable for treatment of bipolar disorders is selected from olanzapine, aripiprazole, quietapine, risperidone, ziprasidone or asenapine, or a pharmaceutically acceptable salt of any of these compounds.
E67. The method according to embodiment 62, wherein said second compound is an antidepressant agent.
E68. The method according to embodiment 67, wherein said antidepressant agent is a serotonin reuptake inhibitor, or any other compound which causes an elevation in the level of extracellular serotonin.
E69. The method according to embodiment 68, wherein said serotonin reuptake inhibitor is a selective serotonin reuptake inhibitor.
E70. The method according to embodiment 62, wherein said antidepressant agent is selected from citalopram, escitalopram, fluoxetine, sertraline, paroxetine, fluvoxamine, venlafaxine, duloxetine, dapoxetine, nefazodone, imipramine, femoxetine, clomipramine, agomelatine, or a pharmaceutically acceptable salt of any of these compounds.
E71. The method according to any of embodiments 62-70, wherein said nalmefene and said second compound are contained in the same unit dosage form.
E72. The method according to any of embodiments 62-70, wherein said nalmefene and said second compound are contained in separate unit dosage forms.
E73. The method according to any of embodiments 52-72, wherein said patient has at least a medium drinking risk level.
E74. The method according to embodiment 73, wherein said patient has a high drinking risk level.
E75. The method according to embodiment 74, wherein said patient has a drinking risk level corresponding to consumption >60 g/day of pure alcohol for men and >40 g/day for women.
E76. The method according to any of embodiments 52-75, wherein said nalmefene is administered as-needed.
E77. The method according to any of embodiments 52-76, wherein said nalmefene is administered in a dose of 10-20 mg such as 10 mg, 11 mg, 12 mg, 13 mg, 14 mg, 15 mg, 16 mg, 17 mg, 18 mg, 19 mg or 20 mg.
E78. The method according to embodiment 77, wherein said nalmefene is administered in a dose of 18 mg.
E79. The method according to any of embodiments 52-78, wherein said nalmefene is administered in the form of a pharmaceutically acceptable acid addition salt.
E80. The method according to embodiment 79, wherein said nalmefene is administered in the form of a hydrochloride salt.
E81. The method according to embodiment 80, wherein said nalmefene is administered in the form of the hydrochloride dihydrate.
E82. The method according to embodiment 81, wherein said nalmefene is administered in a crystalline form.
E83. The method according to any of embodiments 52-76, wherein said nalmefene is contained in an oral dose form such as tablets or capsules.

### EXAMPLES

The invention will be illustrated by the following non-limiting examples.

### Clinical Assessment.

The diagnosis of alcohol dependence was based on the DSM-IV-TR criteria. For this purpose, the investigator interviewed the patient in a structured way by using the Mini International Neuropsychiatric Interview (MINI) standardized interview (Lecrubier et al. The Mini International Neuropsychiatric Interview (M.I.N.I.). A short diagnostic structured interview: Reliability and validity according to the CIDI. European Psychiat. (1997), 12: 224-31). The M.I.N.I. is designed as a brief structured interview for the major Axis I psychiatric disorders in DSM-IV. Its use permits a standardised assessment of the diagnostic criteria. The M.I.N.I. interview was used at the screening visit. Clinicians used it after a training session. The M.I.N.I. approach was used to select patients with a mood disorder at baseline in Example 3.

Another approach to identify patients with a mood disorder is by defining mood disorder at baseline as any ongoing medical history coded by the Medical Dictionary for Regulatory Activities (MedDRA) Preferred Term as ‘Affective Disorder’, ‘Bipolar Disorder’, ‘Depressed Mood’, ‘Depression’, ‘Dysthymic Disorder’, ‘Hypomania’, ‘Major Depression’, ‘Mood Disorder Due To A General Medical Condition’ and/or ‘Seasonal Affective Disorder’. In examples 1 and 2 below patients classified with a mood disorder at baseline was selected based on said MedDRA terms.

### Example 1

### Clinical Efficacy on the Reduction of Alcohol Consumption

The efficacy of nalmefene on the reduction of alcohol consumption in patients with alcohol dependence (DSM-IV) was evaluated in a multi-national, multi-site, randomised, double blind, two parallel group, placebo controlled 1 year safety study (Study 12013A). The efficacy was evaluated over 24 weeks of treatment. The study included outpatients, aged ≧18 years, with a primary diagnosis of alcohol dependence. A patient was eligible for participation in the study if, in the 4 weeks preceding the Screening Visit, he/she had: ≧6 HDDs, ≦14 consecutive abstinent days, did not have serum aspartate aminotransferase (ASAT) and/or serum alanine aminotransferase (ALAT) values >3 times upper limit of the reference range, that are in the investigator's opinion clinically significant. Patients with psychiatric co-morbidity (that is, patients who used stable doses of antipsychotics and/or certain antidepressants) were also included unless the treatment of the psychiatric comorbidity had to take priority over treatment of the drinking problem, or was likely to interfere with study treatment or impairs treatment compliance.

The study included 639 patients, 482 of whom were treated with nalmefene 18 mg in an as-needed dosing regimen. A motivational and adherence enhancing intervention was administered to all the patients to support the patients in changing their behavior and to enhance adherence to treatment.

The efficacy of nalmefene on the reduction of alcohol consumption was measured using two co-primary endpoints: the change from baseline to Month 6 in the monthly number of heavy drinking days (HDDs) and the change from baseline to Month 6 in the average daily total alcohol consumption (TAC). A HDD was defined as a day with a consumption ≧60 g alcohol for men and ≧40 g for women. The change in HDD and TAC over time in patients treated with nalmefene or placebo is reflected in FIGS. 1-2 indicating that the difference between nalmefene and placebo measured in HDDs and TAC at week 24 was more pronounced in the group of patients with a mood disorder at baseline than in patients without a mood disorder at baseline.

### Example 2

### Clinical Efficacy Measured by POMS Score

Assessment of POMs scores in Study 12013A was used to evaluate the effect of nalmefene on mood states and mood changes throughout the study. The change in POMS scores from baseline are illustrated in FIGS. 3-9. FIGS. 3a-9a indicates that in patients without a mood disorder at baseline, the pattern in POMS score was stabile throughout the study with no pronounced difference between nalmefene and placebo.

Tables 7 and 9 indicate that patients with a mood disorder at baseline had higher POMS scores at baseline when compared to those without a mood disorder. FIGS. 3b-9b indicates that the patients with a mood disorder at baseline who received nalmefene had a better POMS score at the end of the study than the patients with a mood disorder at baseline who received placebo. In particular FIGS. 3b, 4b, 5b, 6b and 9b representing total mood disturbance, tension-anxiety, depression-rejection, anger-hostility and confusion, respectively, indicates better POMS scores in weeks 16-24 in patients who received nalmefene compared to patients who received placebo.

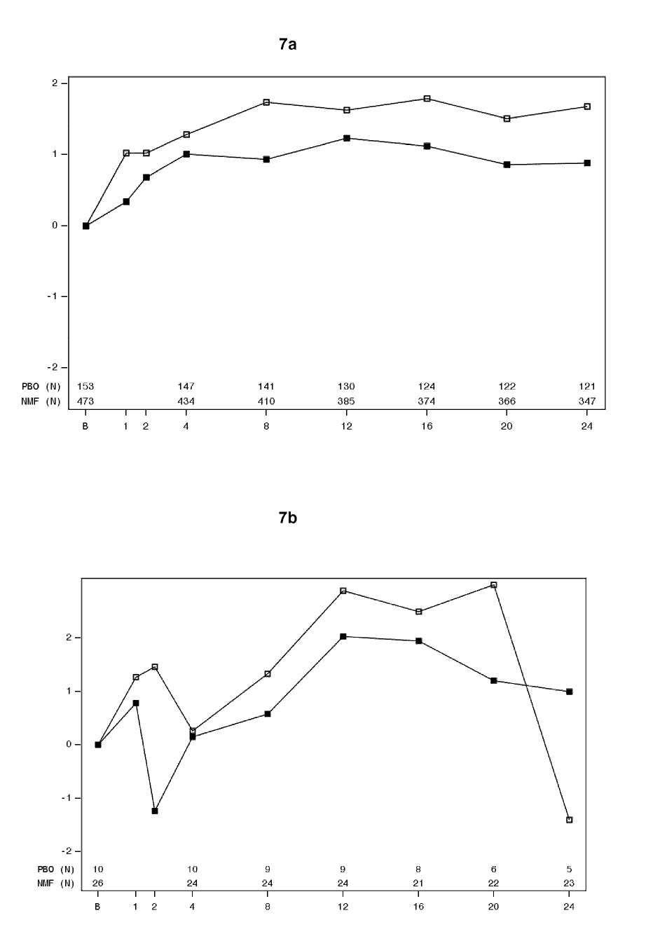

In general, a lower POMS score indicates a better mood state than a higher score except for vigour, illustrated in FIGS. 7a and 7b, wherein a higher POMS score indicates a better mood state.

The demographic data and baseline characteristics for the 12013A study are provided in tables 6-9 below wherein the medical history according to MedDRA was used for patient selection.

TABLE 6
Patient Demographics (APRS) - Patients without a mood disorder at
baseline. Study 12013A

| | | Placebo | Nalmefene | Total |
|---|---|---|---|---|
| Number of Patients | | 156 | 482 | 638 |
| Age | N | 156 | 482 | 638 |
| | Mean | 44.06 | 43.96 | 43.98 |
| | SD | 11.97 | 11.21 | 11.39 |
| | Min | 20.00 | 19.00 | 19.00 |
| | Max | 72.00 | 77.00 | 77.00 |
| | Median | 44.00 | 43.00 | 43.00 |
| Age Group n | <25 | 7 | 14 | 21 |
| | 25-34 | 30 | 88 | 118 |
| | 35-44 | 44 | 155 | 199 |
| | 45-54 | 42 | 143 | 185 |
| | 55-64 | 26 | 56 | 82 |
| | >=65 | 7 | 26 | 33 |
| Sex n | F | 36 | 102 | 138 |
| | M | 120 | 380 | 500 |
| Race n | Asian | 0 | 1 | 1 |
| | Black | 0 | 1 | 1 |
| | Caucasian | 155 | 479 | 634 |
| Race n | Other | 1 | 1 | 2 |

TABLE 7
Baseline Characteristics (APRS) - Patients without a mood disorder
at baseline. Study 12013A

| | | Placebo | Nalmefene | Total |
|---|---|---|---|---|
| Monthly number of HDDs | N | 156 | 481 | 637 |
| | Mean | 13.55 | 13.88 | 13.80 |
| | SD | 6.07 | 6.07 | 6.07 |
| | Min | 6.00 | 5.00 | 5.00 |
| | Max | 28.00 | 28.00 | 28.00 |
| | Median | 12.00 | 12.00 | 12.00 |
| Total Alcohol Consumption | N | 156 | 481 | 637 |
| | Mean | 67.78 | 68.23 | 68.12 |
| | SD | 41.01 | 39.87 | 40.12 |
| | Min | 17.00 | 14.00 | 14.00 |
| | Max | 283.00 | 447.00 | 447.00 |
| | Median | 58.50 | 58.00 | 58.00 |
| POMS - TMD | N | 156 | 482 | 638 |
| | Mean | 40.09 | 38.03 | 38.53 |
| | SD | 41.82 | 35.80 | 37.34 |
| | Min | −25.33 | −20.00 | −25.33 |
| | Max | 206.00 | 161.00 | 206.00 |
| | Median | 29.32 | 29.00 | 29.00 |
| POMS - Tension-Anxiety | N | 155 | 482 | 637 |
| | Mean | 11.83 | 11.86 | 11.86 |
| | SD | 7.66 | 6.56 | 6.84 |
| | Min | 0.00 | 0.00 | 0.00 |
| | Max | 34.88 | 33.00 | 34.88 |
| | Median | 10.00 | 10.75 | 10.00 |
| POMS - Depression Rejection | N | 156 | 482 | 638 |
| | Mean | 14.98 | 13.92 | 14.18 |
| | SD | 14.20 | 12.16 | 12.69 |
| | Min | 0.00 | 0.00 | 0.00 |
| | Max | 60.00 | 58.00 | 60.00 |
| | Median | 10.00 | 10.00 | 10.00 |
| POMS - Anger-Hostility | N | 156 | 482 | 638 |
| | Mean | 10.09 | 10.26 | 10.22 |
| | SD | 8.59 | 8.04 | 8.17 |
| | Min | 0.00 | 0.00 | 0.00 |
| | Max | 36.00 | 42.00 | 42.00 |
| | Median | 8.00 | 8.00 | 8.00 |
| POMS - Vigour | N | 155 | 482 | 637 |
| | Mean | 12.94 | 13.93 | 13.69 |
| | SD | 5.45 | 5.51 | 5.51 |
| | Min | 0.00 | 0.00 | 0.00 |
| | Max | 27.43 | 29.00 | 29.00 |
| | Median | 13.00 | 14.00 | 14.00 |
| POMS - Fatigue | N | 155 | 482 | 637 |
| | Mean | 7.82 | 8.07 | 8.01 |
| | SD | 6.03 | 5.91 | 5.93 |
| | Min | 0.00 | 0.00 | 0.00 |
| | Max | 27.00 | 27.00 | 27.00 |
| | Median | 7.00 | 7.00 | 7.00 |
| POMS - Confusion | N | 156 | 482 | 638 |
| | Mean | 7.69 | 7.84 | 7.80 |
| | SD | 4.89 | 4.55 | 4.63 |
| | Min | 0.00 | 0.00 | 0.00 |
| | Max | 26.00 | 26.00 | 26.00 |
| | Median | 7.00 | 7.00 | 7.00 |

TABLE 8
Patient Demographics (APRS) - Patients with a mood disorder at
baseline (patents selected based on Medical History interviews.
Study 12013A

| | | Placebo | Nalmefene | Total |
|---|---|---|---|---|
| Number of Patients | | 10 | 27 | 37 |
| Age | N | 10 | 27 | 37 |
| | Mean | 47.50 | 49.59 | 49.03 |
| | SD | 12.47 | 10.61 | 11.00 |
| | Min | 18.00 | 26.00 | 18.00 |
| | Max | 61.00 | 67.00 | 67.00 |
| | Median | 48.50 | 51.00 | 51.00 |
| Age Group n | <25 | 1 | 0 | 1 |
| | 25-34 | 0 | 3 | 3 |
| | 35-44 | 3 | 5 | 8 |
| | 45-54 | 2 | 10 | 12 |
| | 55-64 | 4 | 8 | 12 |
| | >=65 | 0 | 1 | 1 |
| Sex n | F | 3 | 14 | 17 |
| | M | 7 | 13 | 20 |
| Race n | Caucasian | 10 | 27 | 37 |

TABLE 9
Baseline Characteristics (APRS) - Patients with a mood disorder at
baseline (patents selected based on Medical History interviews).
Study 12013A

| | | Placebo | Nalmefene | Total |
|---|---|---|---|---|
| Monthly number of HDDs | N | 10 | 27 | 37 |
| | Mean | 15.80 | 17.63 | 17.14 |
| | SD | 5.18 | 7.76 | 7.13 |
| | Min | 11.00 | 8.00 | 8.00 |
| | Max | 28.00 | 28.00 | 28.00 |
| | Median | 15.50 | 16.00 | 16.00 |
| Total Alcohol Consumption | N | 10 | 27 | 37 |
| | Mean | 71.50 | 76.07 | 74.84 |
| | SD | 35.61 | 41.87 | 39.84 |
| | Min | 46.00 | 22.00 | 22.00 |
| | Max | 165.00 | 143.00 | 165.00 |
| | Median | 61.50 | 71.00 | 65.00 |
| POMS - TMD | N | 10 | 27 | 37 |
| | Mean | 59.65 | 47.15 | 50.53 |
| | SD | 41.41 | 45.42 | 44.16 |
| | Min | 7.00 | −6.64 | −6.64 |
| | Max | 124.07 | 128.00 | 128.00 |
| | Median | 57.23 | 31.00 | 37.00 |
| POMS - Tension-Anxiety | N | 10 | 27 | 37 |
| | Mean | 15.10 | 12.96 | 13.54 |
| | SD | 6.98 | 7.78 | 7.54 |
| | Min | 2.00 | 0.00 | 0.00 |
| | Max | 28.00 | 29.00 | 29.00 |
| | Median | 14.50 | 11.00 | 12.00 |
| POMS - Depression Rejection | N | 10 | 27 | 37 |
| | Mean | 20.81 | 18.32 | 18.99 |
| | SD | 14.33 | 14.29 | 14.15 |
| | Min | 2.00 | 3.00 | 2.00 |
| | Max | 41.00 | 55.00 | 55.00 |
| | Median | 22.50 | 12.00 | 13.00 |
| POMS - Anger-Hostility | N | 10 | 27 | 37 |
| | Mean | 12.08 | 10.19 | 10.70 |
| | SD | 13.67 | 8.57 | 10.02 |
| | Min | 0.00 | 0.00 | 0.00 |
| | Max | 41.00 | 29.00 | 41.00 |
| | Median | 8.00 | 8.00 | 8.00 |
| POMS - Vigour | N | 10 | 27 | 37 |
| | Mean | 10.63 | 13.26 | 12.55 |
| | SD | 5.62 | 6.84 | 6.56 |
| | Min | 3.00 | 2.00 | 2.00 |
| | Max | 20.00 | 23.00 | 23.00 |
| | Median | 11.00 | 14.00 | 13.00 |
| POMS - Fatigue | N | 10 | 27 | 37 |
| | Mean | 12.10 | 10.01 | 10.58 |
| | SD | 7.58 | 7.34 | 7.36 |
| | Min | 6.00 | 0.00 | 0.00 |
| | Max | 28.00 | 25.00 | 28.00 |
| | Median | 8.50 | 9.00 | 9.00 |
| POMS - Confusion | N | 10 | 27 | 37 |
| | Mean | 10.20 | 8.93 | 9.27 |
| | SD | 5.43 | 6.35 | 6.07 |
| | Min | 4.00 | 1.00 | 1.00 |
| | Max | 21.00 | 24.00 | 24.00 |
| | Median | 9.00 | 6.00 | 8.00 |

### Example 3

### Alcohol Consumption and POMS TMD in Patients Selected by MINI Inverviews

Table 10 below outlines drinking variables and POMS TMD score patients from the 12013A study with a mood disorder at baseline who were classified based on MINI standardized interviews.

TABLE 10
Drinking variables and POMS TMD score in patients with a mood
disorder at baseline according to MINI assessment.

| | | Nalmefene | Placebo |
|---|---|---|---|
| | N | 22 | 13 |
| HDD | Baseline | 16.0 | 20.5 |
| | 1 year | 6.0 | 13.8 |
| TAC | Baseline | 80.3 | 106.1 |
| | 1 year | 20.8 | 51.1 |
| POMS | Baseline | 68 | 60 |
| | 1 year | 37 | 41 |

### Non-Clinical Assessment.

Further characterization of nalmefene for the treatment of mood disorders can be assessed in non-clinical models e.g. models for assessment of acute effect as outlined in Examples 4-6 and/or models for assessment of chonic effect like the chronic mild stress model described in Example 7. Nalmefene can be assessed in each model both as the sole active substance as well as in combination with a second compound.

Nalmefene can be administered e.g. in the form of nalmefene hydrochloride dissolved in an appropriate amount of saline and dosed to the animals e.g. by subcutaneous administration. A second compound to be combined with nalmefene can be dissolved in an appropriate amount of an appropriate vehicle and dosed to the animals e.g. by subcutaneous administration.

### Example 4

### Forced Swim Test in Mouse

The method, which detects antidepressant activity, follows that described by Porsolt et al (Arch. Int. Pharmacodyn., 229, 327-336, 1977). Mice forced to swim in a situation from which they cannot escape eventually become immobile. Antidepressants decrease the duration of immobility.

Nalmefene was tested as the sole active compound. Mice (NMRI) were individually placed in a cylinder (height=24 em; diameter=13 em) containing 10 cm water (22° C.) from which they cannot escape. The mice were placed in the water for 6 minutes and the duration of immobility during the last 4 minutes was measured. The latency to the first bout of immobility was also recorded starting from the beginning of the test.

10 mice were studied per group. The test was performed blind. Each test substance was evaluated at 3 doses (0.1, 1 and 10 mg/kg), administered s.c. 30 minutes before the test, and compared with a vehicle control group.

Imipramine (32 mg/kg s.c.), administered under the same experimental conditions, was used as reference substance. The experiment therefore included 8 groups.

Data with the test substance were analysed by comparing treated groups with vehicle control using one-way ANOVA followed by post-hoc Dunnett's tests. Data with the reference substance were analysed using unpaired Student's t tests. Due to the number of groups, the study was conducted over 2 separate sub-experiments, each subexperiment including 5 mice per group.

Nalmefene (1 mg/kg), administered s.c. 30 minutes before the test, significantly decreased the duration of immobility, as compared with vehicle controls (−28%, p<0.05) although it did not significantly affect the latency to immobility. It had no effects at 0.1 or 10 mg/kg. Data are further illustrated in Table 11 below.

TABLE 11
Forced swim test in mouse.

| | Duration of immobility (s) | |
|---|---|---|
| Treatment | Mean ± SEM | % compared with vehicle |
| Vehicle | 194 ± 9 | — |
| Nalmefene 0.1 mg/kg | 206 ± 7 | +6% |
| Nalmefene 1 mg/kg | 140 ± 22 | −28% |
| Nalmefene 10 mg/kg | 189 ± 11 | −3% |

### Example 5

### Forced Swim Test in Rats

The method, which detects antidepressant activity, follows that described by Porsolt et al (Eur. J. Pharmacol., 47, 379-391, 1978). Rats forced to swim in a situation from which they cannot escape eventually become immobile. Antidepressants decrease the duration of immobility.

Nalmefene was tested as the sole active compound. Rats (Wistar) were individually placed in a cylinder (height=40 cm; diameter=20 cm) containing 13 cm water (25° C.) for 15 minutes on the first day of the experiment (Session 1) and were then placed back in the water 24 hours later for a 5 minute test (Session 2). The duration of immobility during the 5 minute test was measured. 6 rats were studied per group. The test was performed blind. Nalmefene was evaluated at 3 doses (0.01, 0.1 and 1 mg/kg), administered s.c. 30 minutes before the test, and compared with a vehicle control group.

Data with the test substance were analysed by comparing treated groups with vehicle control using one-way ANOVA followed by post-hoc Dunnett's tests. Data with the reference substance were analysed using unpaired Student's t tests. No significant effect was shown at at 0.01, 0.1 and 1 mg/kg under the given test conditions.

### Example 6

### Marble Burying

The method, which detects anxiolytic/tranquillizing activity, follows that described by Broekkamp et al (Eur. J. Pharmacol., 126, 223-229, 1986). Mice exposed to novel objects (marbles) will bury them in the sawdust floor covering. Anxiolytics decrease the number of marbles buried at non-sedative doses.

Mice (NMRI) were individually placed in transparent plastic cages (33×21×18 cm) with 5 cm of sawdust on the floor and 25 marbles grouped in the centre of the cage. The cage was covered with an inverted plastic cage. Each test cage, together with the marbles, was impregnated with mouse odor before-hand by leaving 10 mice in the cage for 15 minutes. These mice then played no further role in the experiment.

Nalmefene was tested in the marble burying model as the sole active compound. The number of marbles covered by sawdust (⅔ or more) was counted at the end of a 30 minute test. 12 mice were studied per group. The test was performed blind. Nalmefene was evaluated at 3 doses (0.01, 0.1 and 1 mg/kg), administered s.c. 30 minutes before the test, and compared with a vehicle control group. Clobazam (8 mg/kg s.c.), administered under the same experimental conditions, was used as reference substance. The experiment therefore included 8 groups.

Data with Nalmefene were analysed by comparing treated groups with vehicle control using Kruskall-Wallis test followed by Mann-Whitney U tests. Data with the reference substance were analysed using Mann-Whitney U tests. Due to the number of groups, the study was conducted over 2 separate sub-experiments, each sub-experiment including 6 mice per group.

The data showed that nalmefene (0.1 mg/kg), administered s.c. 30 minutes before the test, significantly decreased the number of marbles covered by sawdust, as compared with vehicle controls (−13%, p<0.01). It had no significant effects at 0.01 or 1 mg/kg. Data are further illustrated in Table 12 below.

TABLE 12
Marble burying test in mouse

| | Number of marbles covered by sawdust | |
|---|---|---|
| Treatment | Mean ± SEM | % compared with vehicle |
| Vehicle | 24.8 ± 0.1 | — |
| Nalmefene 0.01 mg/kg | 23.5 ± 1.2 | −5% |
| Nalmefene 0.1 mg/kg | 21.5 ± 1.2 | −13% |
| Nalmefene 1 mg/kg | 22.4 ± 1.4 | −10% |

### Example 7

### Chronic Mild Stress Model

A model for assessment of a cronic effect is the chronic mild stress model which has been described by Willner et al. in Chronic mild stress-induced anhedonia: a realistic animal model of depression. Neurosci Biobehav Rev 1992, 16: 525-534. The test can be performed e.g. as outlined below.

After a period of 2- 3 weeks of adaptation to laboratory and housing conditions, the animals will be first trained to consume the 1% sucrose solution; training will consist of several 1-h baseline tests, in which sucrose solution will be presented, in the home cage, following 14 h food and water deprivation. Subsequently, sucrose consumption will be monitored once weekly, under similar conditions, throughout duration of the study.

On the basis of their sucrose intake in the final baseline test, the animals will be divided into two matched groups. One group of animals will be subjected to the CMS procedure for a period of 8 consecutive weeks. Each week of the stress regime will consist of: two periods of food or water deprivation, two periods of 45-degree cage tilt, two periods of intermittent illumination (light on and off every 2 h), two periods of soiled cage (250 ml water in sawdust bedding), one period of paired housing, two periods of low intensity stroboscopic illumination (150 flashes/min), and three periods of no stress. All the stressors will be of 10-14 h duration and will be applied individually and continuously, day and night. Control non-stressed animals will be housed in separate rooms and will have no contact with the stressed animals. They will be deprived of food and water for 14 h before each sucrose test, but otherwise food and water will be available at libitum.

On the basis of their sucrose intake scores following initial 2 weeks of stress, both stressed and control animals will be further divided into matched subgroups (n=8 per group), and for the subsequent five weeks they will receive daily IP administration of vehicle (0.9% saline, 1 ml/kg), compounds or imipramine HCl (10 mg/kg, IP) as the reference treatments. The volume of all administration will be 1 ml/kg. The drugs will be administered at approx. 10.00 AM and the weekly sucrose tests will be carried out 24 h following the last drug administration. After five weeks, all treatments will be terminated and one additional sucrose test will be carried out following one week of withdrawal. 24 h later blood and brain samples will be collected from all animals (see below) and stored at −70° C. for further biochemical analysis. Stress will be continued throughout the entire period of treatment and withdrawal.

1-15. (canceled)

16. A method of treating a mood disorder comprising administering a therapeutically effective amount of Nalmefene to a patient in need thereof.

17. A method of reducing alcohol consumption comprising administering a therapeutically effective amount of Nalmefene to a patient in need thereof, wherein the patient has alcohol dependence and a co-morbid mood disorder.

18. A method of reducing alcohol consumption and treating a mood disorder comprising administering a therapeutically effective amount of Nalmefene to a patient in need thereof, wherein the patient has alcohol dependence and a comorbid mood disorder.

19. The method of claim 16, wherein the patient also has alcohol dependence.

20. The method of claim 16, wherein said mood disorder is an alcohol induced mood disorder.

21. The method of claim 17, wherein said co-morbid mood disorder is an alcohol induced mood disorder.

22. The method of claim 18, wherein said co-morbid mood disorder is an alcohol induced mood disorder.

23. The method of claim 17, wherein said alcohol dependence is caused by said mood disorder.

24. The method of claim 18, wherein said alcohol dependence is caused by said mood disorder.

25. The method of claim 17, wherein said alcohol dependence and said comorbid mood disorder are not causally related to each other.

26. The method of claim 18, wherein said alcohol dependence and said comorbid mood disorder are not causally related to each other.

27. The method of claim 16, wherein said mood disorder is selected from the group consisting of major depressive disorder, dysthymic disorder, depressive disorder not otherwise specified, bipolar I disorder, bipolar II disorder, cyclothymic disorder and bipolar disorder not otherwise specified.

28. The method of claim 17, wherein said co-morbid mood disorder is selected from the group consisting of major depressive disorder, dysthymic disorder, depressive disorder not otherwise specified, bipolar I disorder, bipolar II disorder, cyclothymic disorder and bipolar disorder not otherwise specified.

29. The method of claim 18, wherein said co-morbid mood disorder is selected from the group consisting of major depressive disorder, dysthymic disorder, depressive disorder not otherwise specified, bipolar I disorder, bipolar II disorder, cyclothymic disorder and bipolar disorder not otherwise specified.

30. The method of claim 18, wherein nalmefene is the sole active ingredient used in the treatment of said co-morbid mood disorder and/or in the reduction of said alcohol consumption.

31. The method of claim 16, wherein said patient is further treated with a second compound which is a mood stabilizer; an antipsychotic agent suitable for treatment of bipolar disorders; or an antidepressant agent.

32. The method of claim 17, wherein said patient is further treated with a second compound which is a mood stabilizer; an antipsychotic agent suitable for treatment of bipolar disorders; or an antidepressant agent.

33. The method of claim 18, wherein said patient is further treated with a second compound which is a mood stabilizer; an antipsychotic agent suitable for treatment of bipolar disorders; or an antidepressant agent.

34. The method of claim 17, wherein said patient has a high drinking risk level.

35. The method of claim 17, wherein the therapeutically effective amount of nalmefene is between 10 and 20 mg.
